(19) United States
(12) Patent Application Publication
(10) Pub. No.: US 2008/0243142 A1
(43) Pub. Date: Oct. 2, 2008

(54) Videotactic and audiotactic assisted surgical methods and procedures

(76) Inventor: Philip L. Gildenberg, Houston, TX (US)

Correspondence Address: VINSON & ELKINS L.L.P., First City Tower, 1001 Fannin Street, Suite 2300, HOUSTON, TX, 77002-6760, US

(21) Appl. No.: 12/070,595

(22) Filed: Feb. 20, 2008

Related U.S. Application Data
(60) Provisional application No. 60/902,229, filed on Feb. 20, 2007.

(51) Int. Cl. A61B 19/00

(52) U.S. Cl. 606/130

(57) ABSTRACT

The present invention provides video and audio assisted surgical techniques and methods. Novel features of the techniques and methods provided by the present invention include presenting a surgeon with a video compilation that displays an endoscopic-camera derived image, a reconstructed view of the surgical field (including fiducial markers indicative of anatomical locations on or in the patient), and/or a real-time video image of the patient. The real-time image can be obtained either with the video camera that is part of the image localized endoscope or with an image localized video camera without an endoscope, or both. In certain other embodiments, the methods of the present invention include the use of anatomical atlases related to pre-operative generated images derived from three-dimensional reconstructed CT, MRI, x-ray, or fluoroscopy. Images can furthermore be obtained from pre-operative imaging and spacial shifting of anatomical structures may be identified by intraoperative imaging and appropriate correction performed.

### CROSS-REFERENCE TO RELATED APPLICATIONS

The present application claims the benefit of co-pending U.S. Provisional Patent Application Ser. No. 60/902,229, filed on Feb. 20, 2007, which is incorporated herein by reference in its entirety.

### TECHNICAL FIELD OF THE INVENTION

This application relates generally to video and audible feedback from 3-dimensional (3-D) imagery, and more specifically to embodiments in which a surgeon is able to access a visual reconstruction of a surgical site and/or receives audible feedback based on the location of a surgical instrument as mapped on reconstructed such surgical views.

### BACKGROUND OF THE INVENTION

Stereotactic surgery is known in the art as a technique for localizing a target in surgical space. The use of stereotactic instrumentation based on tomographic imaging is conventional in surgery. Such methods may involve attaching a localization apparatus to a patient, and then using conventional techniques to acquire imaging data where the data is space-related to the localization apparatus. For example, a surgeon may use an arc system to relate the position of a specific anatomical feature on a patient to a radiographic image. An indexing device, localizer structure or other fiducial apparatus is generally used to specify quantitative coordinates of targets (such as tumors) within the patient relative to the fiducial apparatus.

Current technology also allows use of a frameless system, to provide a visual reference in the operating room. For example, fiducial markers can be placed around an anatomical location or feature of interest so as to be apparent on a pre-operative magnetic resonance imaging (MRI) or computerized tomography (CT) scan. Techniques known in the art can be used in the operating room, usually at the onset of surgery, to localize the fiducial markers located on the patient, and a computer used to compare this information to that from the previous imaging. The actual location of anatomical location or feature of interest may thus be registered to, and correlated with, the computerized three-dimensional reconstruction.

As the surgery proceeds the surgeon can use the image guidance system to locate the surgical target and track a resection, or other instrument's position in space, relative to the target, based on the live-time recognition of fiducial markers located on the instrument itself. Such image guidance systems using visual feedback to the image are disclosed and discussed in more detail in U.S. Pat. No. 5,961,456, incorporated herein by reference. Embodiments disclosed in U.S. Pat. No. 5,961,456 allow the surgeon to observe a video monitor that projects an actual, real-time image of the surgical field and the instrument moving in space. Superimposed on that image is an augmented-reality image, derived from the pre-operative scan, disclosing the position of the target. As the surgery proceeds, the surgeon can use the image guidance system to locate the surgical target. The same guidance system can localize in space the relation of the resection instrument to the target.

A further variation on the above conventional technology is for the surgeon to perform frameless stereotactic surgery with the assistance of an operating microscope that is localized to stereotactic space. The microscope assists enlarged viewing of the surgical field. In this application, the surgeon views a two-dimensional image from the pre-operative scan superimposed on a corresponding three dimensional volume within the surgical field seen directly through the microscope. Although helpful for fine and delicate surgical procedures on microscopic tumors, this technique has limited benefit since the field of view of the microscope is small and microscope programs may not be available at a particular institution. A system using pre-operative scans to guide the surgeon in both microscopically enlarged and unenlarged environments would be highly advantageous.

While serviceable and useful for improved guidance for the surgeon, such prior art visual feedback systems require the surgeon periodically to re-orient his/her field of view from the surgical instrument and the patient to the monitor in order to track the instrument. Recently developed systems, such as that described in U.S. Pat. No. 6,741,883, provide a computer-based system that generates an audible feedback to assist with guidance of a trackable point in space. For example, surgical embodiments include generating audible feedback (to supplement visual and tactile feedback) to a surgeon moving the tip of a probe with respect to a volume of interest such as a tumor.

Over the past decade, endoscopic surgery has become commonplace technique in video-assisted surgery. Endoscopic procedures involve the use of a camera to look inside a body cavity or surgical incision during surgery. These procedures typically consist of a fiber-optic tube attached to a viewing device, used to explore and biopsy internal tissues. One advantage of endoscope assisted surgery is that the miniature cameras used in conjunction with small surgical implements allows exploration and surgical procedures through much smaller than normal incisions making such surgery much less traumatic to the patient than traditional open surgery. For example in laparoscopic surgery, an endoscope is inserted through a small incision in the abdomen or chest, and used to correct abnormalities. In addition, a variety of arthroscopic surgeries are now performed endoscopically on joints such as the knee or shoulder.

Endoscopic techniques are limited, however, by the field of view offered to the surgeon. A visually accessible reconstructed video image of the patient, or a portion thereof, would be extremely advantageous in allowing a surgeon to determine the exact location of endoscopic instruments, the field of view seen with the endoscope, and the proper path to the desired target area. These and other needs in the art are addressed by a computer-based system combining real-time video and 3D reconstructed imagery, potentially in conjunction with audible feedback, to assist with guidance of a trackable point in space.

### SUMMARY OF THE INVENTION

The present invention provides an endoscopic procedure viewing system and method of use. The system of the present invention includes: providing pre-operative scan data representative of a patient's body or part of a patient's body; creating a computer-generated reconstruction of an internal patient volume from the pre-operative scan data; creating a computer-generated real-time image from a video camera or a video camera on an endoscope of at least a portion of the internal patient volume; causing a computer to overlay the computer-generated real-time image and the computer-generated reconstruction with substantial spatial identity and substantial spatial fidelity; and creating computer-generated visual feedback, the computer-generated visual feedback showing position, trajectory and movement of the endoscope in a substantially real-time fashion on the overlay of the computer-generated reconstruction. In certain embodiments, the system of the present invention further includes audible feedback related to instrument and/or endoscope position.

### BRIEF DESCRIPTION OF THE DRAWINGS

For a more complete understanding of the present invention, and the advantages thereof, reference is now made to the following descriptions taken in conjunction with the accompanying drawings, in which the leftmost significant digit in the reference numerals denotes the first figure in which the respective reference numerals appear, and in which:

FIG. 1 schematically illustrates an embodiment in which a patient is being prepared for flexible transesophageal endoscopic surgery assisted by three dimensional pre-operative scan reconstruction and real time video imaging;

FIG. 2 schematically illustrates an embodiment in which a patient is being prepared for endoscopic surgery with a rigid endoscope assisted by three dimensional pre-operative scan reconstruction and real time video imaging;

### DETAILED DESCRIPTION OF THE INVENTION

The present invention provides video and audio assisted surgical techniques and methods. Novel features of the techniques and methods provided by the present invention include presenting a surgeon with a video compilation that displays an endoscopic-camera derived image, a reconstructed view of the surgical field (including fiducial markers indicative of anatomical locations on or in the patient), and/or a real-time video image of the patient. The real-time image can be obtained either with the video camera that is part of the image localized endoscope or with an image localized video camera without an endoscope, or both. In certain other embodiments, the methods of the present invention include the use of anatomical atlases related to pre-operative generated images derived from three-dimensional reconstructed CT, MRI, x-ray, or fluoroscopy.

Certain embodiments of the present invention utilize frameless image guided surgical techniques; however, the present invention also encompasses the use of frame-based image guidance techniques as well. The use of frameless image guided surgery can utilize a system called machine vision. For example, U.S. Pat. No. 5,389,101 discloses a frameless image guidance system. Machine vision typically includes two stereo video cameras overlooking the patient, a portion of the patient or an extremity(s), in addition to the video camera or cameras used to visualize the surgical or endoscopic field. The system of cameras is used to selectively detect fiducial markers and localizes each fiducial in three-dimensional space by triangulation.

The fiducial markers utilized can be any composed of suitable material or be presented in any suitable configuration. One of ordinary skill in the art will readily recognize a wide variety of suitable fiducial markers that can be recognized and registered in three-dimensional (3D) space by an image guidance system. Commonly utilized fiducials include spheres that are approximately 1 cm in diameter or light emitting diodes (“LEDs”).

In addition to the fiducial markers used for triangulation and registration of the video equipment, at least three fiducial markers are typically placed on the patient.

These fiducial markers are visible both on pre-operative images, such as computerized tomography (“CT”) scans or magnetic resonance imaging (“MRI”), intra-operative images (on intra-operative scans) and in real-time by the surgeon by visualization or use of a detection device. The pre-operative and/or intra-operative slice images can be reconstructed into virtual three-dimensional volumetric images that show surfaces, including surface fiducial marks, internal structures, and internal fiducials (if utilized).

In certain embodiments, the locations of the external fiducials affixed to the patient in three-dimensional space are registered by touching an instrument (which is localized in space by attached fiducials allowing the instrument to be localized by machine vision or other localization systems) to each of the fiducials, thus localizing the surface fiducials in space thereby registering the location of the patient to the same stereotactic space being viewed in machine-vision. In alternate embodiments, the external fiducials can be localized in space by video recognition of the imaging system. Alternatively, anatomical details can be used as fiducials by matching a visualization of the surface of the head, face, or body, or internal organs with comparable anatomy contained in the imaging data obtained by preoperative or intraoperative imaging.

Certain embodiments utilize internal fiducials to further aid in registration and localization. Internal fiducials may be localized in space by CT, MRI, ultrasound, x-ray, fluoroscopic or other imaging modality or with an electromagnetic localization system. Surface fiducials can be seen by a video technique, but any technique that visualizes internal anatomy may detect internal fiducials. These fiducials are registered to the same stereotactic space as the fiducials in or on the patient, so the patient and the calibration system are thereby registered to the same stereotactic space.

Alternate image guidance systems can be used in other embodiments of the present invention. For example, laser scanners can be localized to stereotactic space via fiducial markers, then used to scan a patient or portion thereof for registration stereotactic space. One can use stereotactically localized ultrasound or video to register the patient to stereotactic space with any type of such image guidance localizing system.

Alternate embodiments of the present invention include the use image guidance systems other than machine vision. For example certain embodiments utilize an electromagnet system or radiofrequency field to localize fiducials (and hence the patient, the pre-operative virtual images, instruments, video camera, and/or ultrasound transducer) to a predefined stereotactic space. For example, radio frequency interference tags “RFI” may be used as individually identifiable and localizable fiducials, particularly with electromagnetic localization. Fiducials may also be inserted into the body (internal fiducials) and detected with intraoperative imaging. In still other systems, articulating arms or extensions can be used to localize positions with a predefined stereotactic space. The use of RFIs also allows each fiducial to be specifically recognized and localized. For example, a tracking system can be employed that recognizes a particular instrument by the frequency or identification code of its fiducial.

Certain embodiments of the present invention also include a calibration system. In such embodiments, a number of fiducials at predefined locations from each other are localized in the defined stereotactic space.

In some embodiments, a video camera is also localized in the predefined stereotactic space with the image guidance system of choice. This video camera can be used to scan external surfaces of the patient for registration to the stereotactic space in real-time video or as pre- and intra-operative digital pictures. For example, U.S. Pat. No. 7,130,717, which is hereby incorporated by reference, describes the use of a frameless image guidance system in conjunction with a separate video camera to scan a patient's head prior to robotically assisted hair transplant surgery. In alternative embodiments, a localized video camera or other digital camera can be used to capture stereo or multiple still images to reconstruct a three dimensional map of the surface. While in still other embodiments, two video cameras can be used to acquire a stereo three-dimensional map of the patient surface to register to stereotactic space.

In certain embodiments, intra-operative scans or images are also registered to the predefined stereotactic space and can be used to verify anatomical locations and patient position. For example, intra-operative images and/or scans can be used to update images to reflect a change in position of internal structures or organs with respect to body position, retraction, as resection progresses, or with respiratory movements. Such intra-operative scans or images include, but are not limited to, x-ray images, fluoroscopy or ultrasound images. For example, an ultrasound transducer can be localized with the same registration system used by any image guidance technique to determine the ultrasound transducer's position in relation to the patient, and subsequently register the two- or three-dimensional ultrasound images to the patient. Another exemplary use would involve fluoroscopic or x-ray images of a patient's spine for registration and incorporation in the defined stereotactic space allowing for the spine to be displayed in a 3D reconstructed image.

Those of skill in the art will appreciate that the types of imaging or scanning techniques described are exemplary only and that the present invention encompasses the use of any presently used or future imaging or scanning system that can provide data for incorporation into the visual displays discussed herein.

The present invention also provides for the visual overlay of the real-time video (or pre- and intra-operative still photos) with the predefined stereotactic space defined by the image guidance system. 3D reconstructions of the patient based on pre-operative scans and imaging can also be presented in the visual overlay (compilation). Such 3D reconstructions can be used to display target tissue volumes and anatomical structures, or internal or external fiducials, or instruments in or around the surgical field, or implantable devices such as used in spinal surgery. In certain embodiments, the present invention further provides representations of an implantable device to determine proper insertional position and trajectory/path, as well as device size.

In addition, in certain embodiments a digital anatomical atlas can also be incorporated into the video compilation. In such embodiments, intra-operative (or pre-operative) images and/or scans can be merged with images from the digital atlas to distort or reconfigure the atlas to more closely resemble the actual dimensions of an individual patient and provide anatomical identification of structures.

This use of a stereotactic image guidance system during an endoscopic procedure provides the surgeon with an enhanced visual input. In certain embodiments of the present invention, the video-camera used to relay real-time images can be an endoscopic camera. In still others, an endoscopic camera is utilized in addition to an external real-time video camera. In certain such embodiments, the real-time video represents the surgeon's-eye-views (reproduces the surgeon point of view or an approximation thereof).

During endoscopic procedures, a surgeon normally has an extremely limited visual field. For example, in typical endoscopic procedures, the surgeon is looking though a video portal on the endoscope or is watching a video-monitor that displays the endoscopic image. The visualized field, therefore, is limited or restricted to that captured by the endoscope. Adding the endoscopic image to the video compilation described above provides the surgeon with a myriad of positional references during a procedure. The surgeon is able to assess the relative position of the endoscope with respect to the 3D reconstructed images of the patient from pre-operative scans/images. This allows the surgeon to determine the location of the tip of the endoscope and the field of vision with respect to targeted tissue, and internal organs/anatomically locations, essentially allow the surgeon access to an expanded visual field. The field of view can be displayed on a virtual image of an anatomical or pathological structure by a highlighted area, a cursor, or any such indicator.

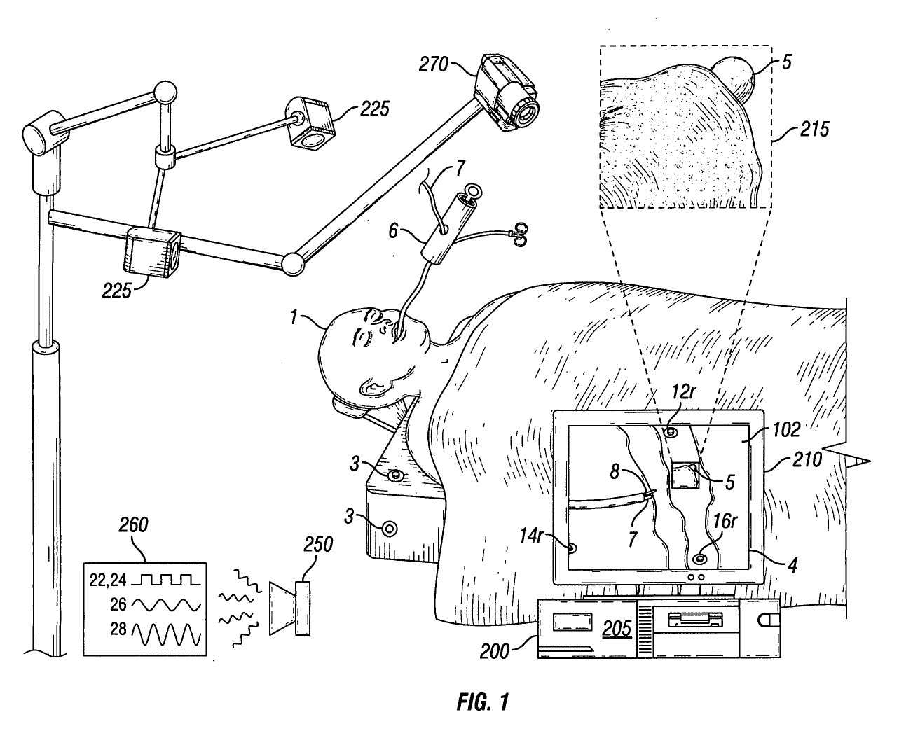

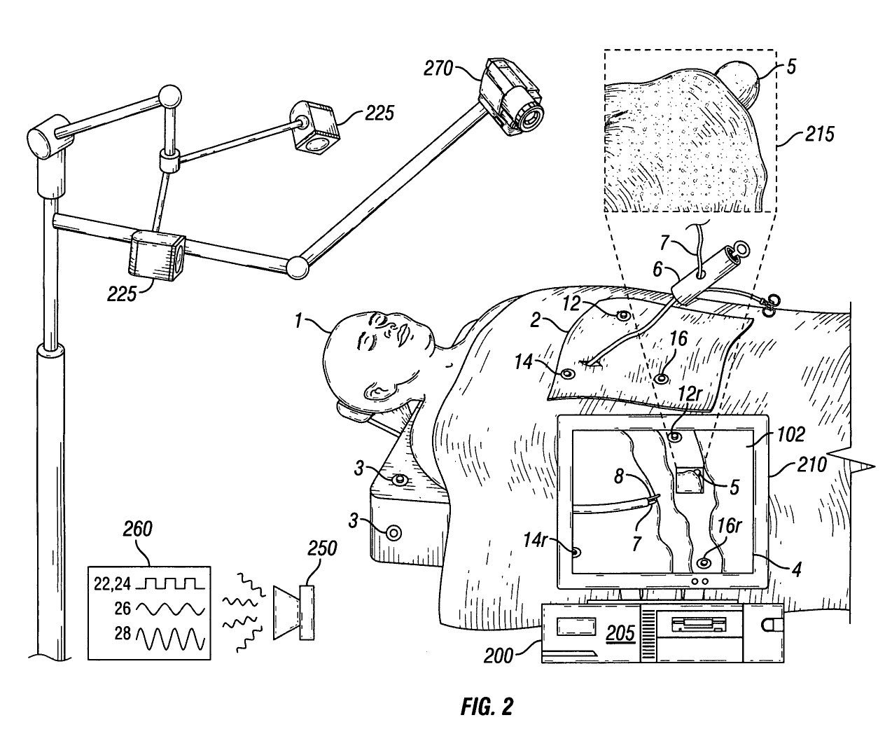

FIGS. 1 and 2 illustrate schematically an embodiment of the present invention in which an endoscopic procedure is performed with stereotactic video assistance. It will be appreciated that the present invention is not limited to the particular embodiment depicted in FIGS. 1 and 2. It will be further appreciated that embodiments are possible for a multitude of procedures in which it is advantageous to use video to monitor and/or guide, substantially in real-time, the location of an endoscope, probe and/or other workpoint in relation to a field of work.

FIGS. 1 and 2 schematically illustrate a patient 1 who is prepared for one embodiment of an endoscopic stereotactic-assisted surgical procedure as disclosed in this application. FIG. 1 depicts an esophageal endoscopic procedure, while FIG. 2 depicts endoscopic entry via a surgical opening. Surrounding the external surgical field 2 are fiducial markers 12, 14, and 16. System registration fiducial markers 3 can be used to register the stereotactic space defined by the stereotactic cameras 225 and serve as a calibration system. The video camera 270 is imaging the external surgical field 2, which represents the surgeon's eye-view, the localization of which is based on the positions of internal or surface fiducials. Typically, the camera 270 would be sterile and suspended, with a malleable bracket, within the surgical field and localized by fiducials localized by the same machine vision, (rather than necessarily visualized fiducials) so it is localized to the same stereotactic space as everything else. Alternatively, the video image or images of the intended operative field may be supplied by the video camera or cameras which are part of the exoscope system. The 3D reconstructed image 4 displayed on the monitor 210 is generated based on pre-operative scans and images. As shown, display 4 is a 2-dimensional monitor. One can also use a 3D video display with appropriate glasses or a pair of uni-ocular video displays. The 2D slices as pictured represent a slice orthogonal to the line-of-sight at a depth selected by the surgeon to demonstrate the outline of the structure at the depth being addressed surgically. The 3D reconstructed image 4 also depicts the locations of fiducial markers 12, 14, and 16 (shown on the reconstructed image as 12r, 14r, and 16r) based on position in the pre-operative scans/images. Overlaying the 3D reconstructed image 4 can be a transparent or translucent image from the video camera 270 in the surgical field verifying the fiducial marker locations 12r, 14r, and 16r. The image guided camera need not visualize the fiducials, but gets its localization from fiducials attached to the camera and visualized by the machine vision or other localizing system.

It will be understood that numerous fiducial marker systems are known in the art and that the number of fiducial markers used may vary as appropriate. Some systems attach the fiducial markers directly to the patient, an example of which is illustrated in FIG. 1. Other systems, examples of which are not illustrated, may use frame-based stereotactic systems which are well-defined in the prior art. It will be understood that the present invention is not limited to any particular type of fiducial marker system.

FIG. 1 schematically illustrates a target tissue 5 as the item or feature of interest in this embodiment. It will be appreciated that the present invention is not limited in this regard. The item of interest may be any point, object, volume and/or boundary in three-dimensional space in reference to which video representations would be advantageous to help guide probes and/or other instruments in the space. It will be appreciated that the depicted endoscopic application of the technology is only one embodiment and that such techniques may be applied to other surgical and/or non-surgical fields, as well. The localization system may localize a video camera peering into the surgical field, an operating microscope or stereoscope visualizing the surgical field, or a conventional or stereoscopic endoscope. In addition, the same localization system may localize one or several surgical instruments and any virtual images reconstruction from preoperative or intraoperative scans. Since all of the above would be localized to the same localization system, they would also be localized to each other.

FIGS. 1 and 2 further depicts a computer system 200 includes a processor 205 and a monitor 210. It will be understood that the computer system 200 can generate and display the 3D reconstructed image 4 of the patient according to 3D resolution of the series of layered images 102 acquired earlier and described above with reference to FIGS. 1 and 2. It will also be understood that the monitor 210 can further display a view 215 comprising an enlarged 3D zone of such a computer-generated 3D reconstructed image 4. The view 215 may also be computer generated images of anatomy obtained from an integrated digital anatomical atlas. It will be seen on FIGS. 1 and 2 that the view 215 displayed on the monitor 210 is only a partial view of the patient 1, wherein a surgical field including the target tissue 5 (for example a gastric tumor) is enlarged. Computerized techniques well-known in the art will be able to enlarge or reduce the magnification of the reconstruction of the layered images 102 and display same on the monitor 210.

It will be appreciated that the present invention is not limited to any particular computer system 200. Computer systems are known in the art, both stand-alone or networked, having the processing functionality to generate 3D reconstructive images resolved from a series of layered views, and then to enlarge, rotate and/or generally manipulate the reconstructive image on a display, and to integrate, overlay or fuse images obtained from several different imaging sources or anatomical atlas. Examples of a suitable computer system 200 in current use include systems produced by Radionics/RSI of Burlington, Mass., or the Stealth Image Guided System produced by the Surgical Navigation Technology Division of Medtronic in Broomfield, Colo.

Alternatively (not illustrated), computer graphics images, based on imaging data, may be placed in the direct view field of a surgical microscope. For example, see U.S. Pat. No. 4,722,056 granted Jan. 26, 1988 to Roberts et al. Stealth Image Guided System produced by the Surgical Navigation Technology Division of Medtronic in Broomfield, Colo. also makes a system whose capability includes importing a reconstructed graphics image into a “heads-up” display seen concurrently with the surgical field, either directly or through a surgical microscope.

Looking at the view 215 on the monitor 210 in FIG. 1 more closely, it will be understood that prior to surgery, the computer system 200 will have been coded to define and/or identify zones of interest visible in the 3D image reconstructive 4 based on localizations in the pre-operative scans and images, or a digital atlas. These zones of interest may include points, volumes, planes and/or boundaries visible on the 3D reconstructive image 4 and enlargement 215 and differentiable (able to be differentiated and/or distinguished) by the computer system 200. In the case of the example shown on FIG. 1, the computer system 200 has been previously coded to define and identify at least two volumes and one 3D boundary: the target tissue 5; healthy gastric tissue; and a boundary between the target tissue 5 and the healthy tissue.

Digital output signals from the cameras 225 and 270 are received by the computer system 200 (connections omitted for simplicity and clarity). The computer system 200 then resolves, using conventional computer processing techniques known in the art, the cameras' signals into a computer-generated combined “stereo” 3D view of the patient or surgical field.

Although FIG. 1 shows only one visualizing camera 270 and two localizing cameras 225 for simplicity and clarity, it will be appreciated that multiple additional cameras may be included. As is well understood in the art, the greater the number of cameras that are provided viewing the patient 1, the more sophisticated and detailed a “stereo” 3D view of the patient may be obtained by concurrently resolving such multiple cameras' views.

With further reference to FIG. 1, an endoscope 6 is provided to the surgeon for use in an endoscopic procedure. Although the endoscope may be introduced orally, as shown, it much more commonly is introduced through a small skin incision or port near the target or into the body cavity housing the target. Most endoscopes are rigid, but some are flexible, as shown. The rigid scope may be localized by fiducials attached externally where they might be localized by machine vision or localized by either internal or external fiducials if they are localized in an electromagnetic field. In order to localize a flexible endoscope with external fiducials, it would be necessary to have a built-in system to identify where and how the endoscope is flexed thus indirectly determining the position of the distal end of the endoscope. Alternatively, the flexible endoscope may have fiducials near its tip that can be localized by intraoperative imaging or an electromagnetic field, and indicate the position and trajectory of the tip of the flexible endoscope. Those of skill in the art appreciate that endoscopes can be used in a wide variety of surgical procedures and the present invention is not limited to the example depicted in FIG. 1 or 2.

In addition, stereoscopic endoscopes can be utilized in the present invention. Stereo-endoscopes provide depth perception with a three-dimensional view of the field, the virtual image can be displayed according to the perspective of each eye-piece on such endoscopes. The virtual image is already a three-dimensional volume, and can be displayed as such in each eye-piece or monitor of the stereoscopic endoscopic display, thereby giving the virtual image the perception of being three-dimensional, as well. Furthermore, currently available stereo-endoscopes, such as the DaVinci robotic system, can be incorporated into the present invention. In such embodiments, the videoscopic surgery can be stereoscopic, but that image can be used to guide the positioning of the robotic visualization system by commanding the robot appropriately. In addition, the position of the endoscope and the working ports, used to introduce surgical instruments into the endoscopic surgical field, can be adjusted by the control system of the DaVinci or other robotic surgical system according to the localization information provided by the techniques described herein. That is, the endoscope may be positioned by hand and the position monitored and corrected by the image guidance system, or the same image guidance system may be used to determine the ideal position and trajectory of the endoscope and working ports that are attained by robotic control. In certain embodiments, the positioning mechanism of the DaVinci endoscope arm can be fed into the data base containing the patient's localization and the view of the DaVinci stereo-endoscope indicated in the virtual image, or the patient localization data can be used to position the DaVinci endoscope arm manually or robotically.

It will be understood by those of ordinary skill in the art, that the present invention can be used with any number of surgical robotic systems and used guide any such robotic system in an endoscopic channel. Furthermore the videotactic systems of the present invention can be used to register and guide a robot or surgeon in a working surgical channel or channels, and are therefore not limited to the positioning of the endoscope 6 itself.

The endoscope 6 includes an endoscopic camera 7, and an instrument or resection device 8 on the end for use by the surgeon in excision of the target tissue 5. The endoscope 6 includes at least three fiducial markers to register the position and trajectory of the endoscope 6 for incorporation into image compilation (image overlay) 102. Typically tracking and localization of the proximal end of the endoscope, via registration of its fiducials, will indirectly indicate the localization of the distal end of the endoscope, its trajectory, its line-of-sight, and consequently its field of view resection device 8, although the present invention is not limited in this regard. Again, the number of fiducial markers used may vary as appropriate. The mechanism may comprise any type of source disposing the resection device 8 to be trackable, including various forms of electromagnetic radiation, radio frequencies and/or radioactive emissions, and the like. The incorporation of the endoscope 6 into the 3D reconstructed image 4 aids the surgeon during insertion of the endoscope 6 by providing visual feedback of the endoscope's progress with respect to internal organs and other anatomical features. For example, the monitor 210 can display the surface of organs with the location being visualized by the endoscope highlighted. Furthermore, the computer can automatically calculate the distance from the distal end of the endoscope to any organ displayed in the 3D reconstructed image 4 as well show the location of blood vessels and nerves to be avoided.

In certain embodiments, the endoscopic camera 7 provides an endoscope-eye-view that is incorporated into the reconstructed image 4 and/or the enlargement 215. Furthermore, the images provided by the endoscopic camera 7, the pre-operative scans, intraoperative scans, and/or digital atlases can be used to generate and display an instrument-eye-view within the reconstructed image 4 and the enlargement 215. The instrument-eye-view can thus display a point of view of the instrument as it approaches a target structure, as well as display the instruments path.

As depicted in FIG. 1, the cameras 225 track the fiducial markers on the endoscope 6, and allow the locus of the resection device 8 to be determined by the computer system 200. Thus, the computer-generated stereo 3D view of the surgical field based on the combined views of the cameras 225, with the 3D view based in part on the pre-operative scans and images, and with the localization based on the combined views of the cameras, will further include the locus of the resection device 8. Endoscope cameras are commonly at the proximal end or outside of the scope, which is a fiber-optic system to deliver the image from beyond the tip of the endoscope to the camera. Alternatively, the camera may be a miniaturized camera that is threaded into the endoscope or a channel of the endoscope to its tip and see the field-of-view directly, although that is presently rare and generally still under development. During resection or other manipulation of tissue that constitutes the purpose of the surgery, the endoscope camera typically shows the tip or working end of the instrument and the target tissue immediately surrounding it.

It will be appreciated that the present invention is not limited to any type of instrument used by the surgeon in generating a trackable tip of the endoscope. Although the embodiment of FIG. 1 depicts a biopsy or resection instrument 8, the instrument used by the surgeon may be any suitable instrument upon which a trackable point or points may be deployed, such as a resection or excising instrument, a means of coagulating tissue or blood vessels, a means of cutting or incising tissue, a means of injection a substance, a means of occluding blood carrying or other vessels, a means of anastomosis of structures or securing tissue or applying sutures or other fastening devices, or other instrument. Indeed, it will be further appreciated that the present invention is not limited to use of a surgical instrument, or location of a trackable point on a tip, or confinement to one instrument and/or trackable point. Depending on the application and the deployment of the present invention, any number of instruments and/or trackable points may be used. Further, the trackable points may be deployed at any desired position with respect to the instruments. Moreover, in embodiments where multiple trackable points are used, as long as different trackable points are disposed to exhibit different tracking signatures that are differentiable by the cameras 225 or other detectors, it will be appreciated that the computer-generated stereo 3D view of the patient 1 based on the combined views of the cameras 225 may also include a separate locus for each of such different trackable points. Furthermore, it will be understood that multiple endoscopes 6 or instruments can be utilized and incorporated into the 3D reconstructed image 4.

Tracking and registration of the surgical instrument of choice to the defined stereotactic space has the further advantage of allowing for the integration of the physical dimensions of specified surgical instrument or device into the volumetric planning of the surgery. The planning can include depicting various surgical instruments into the virtual reality created by the 3D reconstructed image 4. Furthermore, similar techniques can be utilized to provide volumetric analysis for implantable devices. Virtual simulations of various implantable devices, such as screws, rods and plates for spinal fusion or bone fixation, electrodes, and catheters, can be incorporated into the 3D reconstructed image 4 in order to determine proper size and positioning. Once determined, intra-operative scans/images can be used to verify proper and precise placement of such implantable devices. Those of skill in the art will readily recognize that the present invention can be used to register, track and plan any of the multitude of instruments or devices that might be utilized in a wide variety of endoscopic, minimally invasive, or other surgical procedures. For example, the present invention can be used to determine the proper size of and placement of retractors, externally or internally.

Returning to FIG. 1, the computer system 200 now overlays the computer-generated stereo 3D view of the patient 1 (based on the combined views of the cameras 7, 270 and 225), with the computer-generated 3D reconstructed image 4 according to 3D resolution of the series of layered images 102 (based on the pre-operative scan described above with reference to FIG. 1). Computer system 200 advantageously uses the fiducial markers 12, 14, and 16 to coordinate and match the overlay of the computer-generated stereo 3D view and the computer-generated 3D reconstructed image 4. An intraoperative image, such as that obtained from CT, MRI, x-ray, fluoroscopy or ultrasound can be used to correct the spatial distortion or localization of tissues that may have shifted, moved, or become distorted since the original pre-operative images had been obtained. The image guided ultrasound image can be used to identify any shift, displacement or distortion of the internal anatomy in comparison with that obtained from the pre-operative imaging studies, and that image is shifted or distorted to correspond to the actual position of anatomical structures during surgery, so that those corrected images can be used to create the virtual image or target points for surgical localization. Reference can be made to anatomical structures and/or to internal fiducials to obtain the data required for such corrected reconstruction.

Once the computer-generated stereo 3D view and the computer-generated 3D reconstructed image 4 are coordinated, the computer system 200 may then relate the locus of the resection device 8 of the endoscope 6, as tracked by the cameras 225, to the previously-coded zones of interest on the 3D reconstructed image 4. Specifically, in the example depicted in FIG. 1, the computer system 200 will be able to use fiducial markers 12, 14 and 16 and the fiducial markers on the endoscope 6 to triangulate the resection device 8, as tracked by the cameras 225, and then pinpoint the current position of the resection device 8 with respect to the previously-coded zone or zones of interest, or target tissue 5 on the computer-generated 3D reconstructed image 4 and 215. The tracking and registration of the surgical instrument, such as resection device 8 in FIG. 1, furthermore allows the computer 200 to calculate and display distances and vectors between the resection device 8 and any structure of interest, such as the targeted tissue 5.

Certain embodiments of the present invention further include an audible feedback component. FIG. 1 shows a loudspeaker 250 that is provided to enable the computer system 200 to give an audible feedback 260 to the surgeon according to the position of the resection device 8 (or any other surgical instrument) with respect to the previously-coded zone or zones of interest on the 3D reconstructed image such as the target tissue 5. In the example depicted in FIG. 1, it will be seen that when the resection device 8 is at positions 22 and 24, as shown on the monitor 215, the computer system 200 detects the resection device 8 to be at the boundary of the target tissue 5, and generates an audible feedback 260 comprising a buzz sound typical of a square wave, as indicated in FIG. 1 by the square wave shown in the audible feedback 260 associated with position numbers 22 and 24. When the resection device 8 is at position 26, the computer system 200 detects the resection device 8 to be in the target tissue 5, and generates an audible feedback 260 comprising a pure tone typical of a sine wave, as indicated in FIG. 1 by the lower frequency, lower amplitude sine wave shown in the audible feedback 260 associated with position number 26. When the resection device 8 is at position 28, the computer system 200 detects the resection device 8 to be outside of the target tissue 28, and generates an audible feedback 260 comprising a different (higher) tone, as indicated in FIG. 1 by the higher frequency, higher amplitude sine wave shown in the audible feedback 260 associated with position number 28.

Thus, the surgeon may receive audible feedback as to the position of an instrument with respect to a volume and/or boundary of interest within an overall surgical field. The surgeon may then use this audible feedback to augment the visual and/or tactile feedback received while performing the operation.

It will be appreciated that the present invention is not limited to the types of audible feedback described in exemplary form above with respect to FIG. 1. Consistent with the overall scope of the present invention, different audible feedbacks may vary in tone, volume, pattern, pulse, tune and/or style, for example, and may even include white noise, and/or pre-recorded or computer generated utterances recognizable by the surgeon. In other embodiments, the audible feedback may be substituted for, and/or supplemented with, a complementary tactile or haptic feedback system comprising a vibrating device (not illustrated) placed where the surgeon may conveniently feel the vibration. Different audible feedbacks may be deployed to correspond to different types of vibratory feedback, including fast or slow, soft or hard, continuous or pulsed, increasing or decreasing, and so on. In various illustrative embodiments, for example, a steady tone could indicate that the zone of interest is being approached, with the pitch increasing until the border of the zone is reached by the dissection instrument and/or pointer, so the highest pitch would indicate contact with the zone or zones of interest. Furthermore, when the tip of the instrument lies within the zone or zones of interest, an interrupted tone at that highest target pitch could be heard, with the frequency of the signal increasing until becoming a steady tone when the border is reached.

It will be further appreciated that the present invention is not limited to embodiments where the audible feedback is static depending on the position of a trackable point with respect to predefined zones of interest. Dynamic embodiments (not illustrated) fall within the scope of the present invention in which, for example, the audible feedback may change in predetermined and recognizable fashions as the trackable point moves within a predefined zone of interest towards or away from another zone of interest. For example, if the audible feedback 260 on FIG. 1 comprises silence for all positions on the boundary of the target tissue 5 (including the positions 22 and 24), a pure sine wave tone for all positions in the target tissue 5 (including the position 26) and a square wave “buzz” for all positions outside the target tissue 5 (including the position 28), according to an exemplary dynamic embodiment (not illustrated), the computer 200 might be disposed to increase the pitch of the sine wave tone and the square wave “buzz” as the position of the resection device 8 moved closer to the boundary of the target tissue 5. Thus, the surgeon would be able to interpret the dynamic audible feedback in a yet further enhanced mode, in which both pitch and type of sound could be used adaptively to assist movement and/or placement of an instrument in the surgical field. Another illustrative system embodiment might involve intermittent pulsatile and/or pulsating sounds when the resection device 8 lies within the target tissue 5, with the rate of pulsation increasing as the boundary of the target tissue 5 is approached so the pulsation rate becomes substantially continuous at the boundary of the target tissue 5 and then silent outside the defined volume.

Of course, other dynamic variations on audible feedback are possible, such as changes in volume, and/or changes in predetermined utterances. These other variations may be substituted for the changes in pitch and/or type suggested above, and/or may supplement the same, to enhance yet further the audible feedback by making the audible feedback more multi-dimensional.

Furthermore, those of skill in the art will recognize that the audible feedback of the present invention is not limited to use in identifying the boundaries of a structure of interest. The audible feedback can be utilized to provide feedback to the surgeon for a wide variety of activities in which position and movement are integral. For example, the audible feedback can be set to provide input to the surgeon based on maintaining the insertion of the endoscope on a predefined vector, or for the proper implantation position of internal devices.

Those of skill in the art will also appreciate that the computerized aspects of the present invention may be embodied on software operable on a conventional computer system, such as those commercially-available computer systems described above, or, alternatively, on general purpose computers standard in the art having at least a processor, a memory and a sound generator. IBM, Dell, Compaq/HP, Sun and other well-known computer manufacturers make general purpose processors for running software devised to accomplish the computerized functionality described herein with respect to the present invention. Conventional or graphics intensive software languages, such as UNIX and C++, well-known to be operable on such general purpose machines, may be used to create the software.

Although the present invention and its advantages have been described in detail, it should be understood that various changes, substitutions and alterations can be made herein without departing from the spirit and scope of the present invention as defined by the appended claims.

What is claimed is:

1. An endoscopic procedure viewing system, the system comprising:
(a) providing pre-operative scan data representative of a patient's body or part of a patient's body;
(b) creating a computer-generated reconstruction of an internal patient volume from the pre-operative scan data and/or digital atlases;
(c) creating a computer-generated real-time image from a camera on an endoscope of at least a portion of the internal patient volume;
(d) causing a computer to overlay the computer-generated real-time image and the computer-generated reconstruction with substantial spatial identity and substantial spatial fidelity; and
(e) creating computer-generated visual feedback, the computer-generated visual feedback showing position, trajectory and movement of the endoscope in a substantially real-time fashion on the overlay of the computer-generated reconstruction.

2. The system of claim 1, wherein (c) and (d) are performed using a system of fiducial markers.

3. The system of claim 2, wherein the fiducial markers are light emitting diodes.

4. The system of claim 2, wherein (c) and (d) are performed using a marker system selected from the group consisting of:
(1) spherical objects;
(2) radio frequency tags; and
(3) light emitting diodes.

5. The system of claim 1, wherein the computer-generated reconstruction is generated in part by resolving a series of layered images.

6. The system of claim 5, wherein the layered images are selected from the group consisting of:
(1) computerized tomography (CT);
(2) magnetic resonance imaging (MRI);
(3) x-ray;
(4) fluoroscopy;
(5) ultrasound; and
(6) proton beam imaging.

7. The system of claim 1, further comprising:
(f) creating a computer-generated reconstruction of an internal patient volume from the pre-operative scan data, the computer-generated reconstruction identifying at least one feature of interest within the overall volume; and
(g) causing the computer to track the endoscope-eye-view with substantial positional fidelity to the computer-generated real-time image.

8. The system of claim 1, further comprising incorporating intra-operative scan data into the computer-generated reconstruction.

9. The system of claim 1, the computer-generated reconstruction further includes data from a digital atlas.

10. The system of claim 1, further comprising a digital representation of an implantable device in the computer-generated reconstruction.

11. A method of use of the system of claim 10, wherein the system is used to display the digital representation of the implantable device in various positions or to display the path to insertion for the implantable device.

12. A method of use of the system of claim 10, wherein the system is used to determine proper size of the implantable device.

13. An endoscopic viewing system for providing visual and audible feedback, the system comprising:
(a) providing pre-operative scan data representative of a patient's body;
(b) creating a computer-generated reconstruction of an internal patient volume from the pre-operative scan data and/or digital altases, the computer-generated reconstruction identifying at least one feature of interest within the overall volume;
(c) creating a computer-generated real-time image from a camera on an endoscope of at least a portion of the internal patient volume, the computer-generated real-time image further including at least one trackable point, the at least one trackable point movable in real-time with respect to the overall volume;
(d) causing a computer to overlay the computer-generated real-time image and the computer-generated reconstruction with substantial spatial identity and substantial spatial fidelity; and
(e) creating computer-generated visual feedback, the computer-generated visual feedback showing movement of the endoscope in a substantially real-time fashion on the overlay of the computer-generated reconstruction;
(f) causing the computer to track the at least one trackable point with substantial positional fidelity to the computer-generated real-time image; and
(g) creating computer-generated audible feedback, the computer-generated audible feedback describing movement of the at least one trackable point with respect to the at least one feature of interest.

14. The method of claim 13, wherein (d), (e) and (f) are performed using a system of fiducial markers.

15. The system of claim 13, wherein (d), (e) and (f) are performed using a marker system selected from the group consisting of:
(1) spherical objects;
(2) radio frequency tags; and
(3) light emitting diodes.

16. The method of claim 13, wherein the computer-generated audible feedback comprises at least one type of sound selected from the group consisting of:
(1) a tone;
(2) a buzz;
(3) a tune;
(4) white noise;
(5) a pre-recorded or computer generated utterance;
(6) substantial silence; and
(7) an intermittent pulsatile tone; and
(8) a variable vibrating signal.

17. The method of claim 13, wherein the computer-generated audible feedback comprises at least one variation selected from the group consisting of:
(1) pitch variation;
(2) volume variation;
(3) pulse variation;
(4) type of sound variation; and
(5) utterance variation.

18. The method of claim 13, wherein the at least one trackable point is a tip of a surgical instrument on the endoscope, and at least one other point so that the trajectory of the instrument can be determined, or the trajectory can be determined directly by relting it to the orientation of the localization fiducials.

19. The method of claim 13, wherein the computer-generated reconstruction is generated in part by resolving a series of layered images.

20. The method of claim 19, wherein the series of layered images is obtained using a process selected from the group of:
(1) computerized tomography (CT);
(2) magnetic resonance imaging (MRI);
(3) fluoroscopy and,
(4) ultrasound.

21. The system of claim 13, further comprising a digital representation of an implantable device in the computer-generated reconstruction.

22. The system of claim 13, further comprising creating and displaying at least a portion of the computer generated real time image that represents an instrument-eye-view.

23. The system of claim 22, wherein the instrument-eye-view is displayed as a highlighted area or cursor.
